(19) United States
(12) Patent Application Publication
(10) Pub. No.: US 2025/0097648 A1
(43) Pub. Date: Mar. 20, 2025

(54) HEARING SYSTEM AND METHOD FOR GENERATING MONAURAL OR BINAURAL BEATS

(71) Applicant: Sivantos Pte. Ltd., Singapore (SG)

(72) Inventors: Matthias FRÖHLICH, Erlangen (DE); Jens HAIN, Erlangen (DE); Florian HÖGL, Erlangen (DE); Frank NAUMANN, Erlangen (DE)

(21) Appl. No.: 18/889,645

(22) Filed: Sep. 19, 2024

(30) Foreign Application Priority Data
Sep. 19, 2023 (DE) 10 2023 209 094.5

(51) Int. Cl. H04R 25/00

(52) U.S. Cl. CPC H04R 25/552; H04R 25/554; H04R 25/70

(57) ABSTRACT

A monaural or binaural hearing system is configured to generate monaural or binaural beats and includes at least one hearing device having a main signal path. The hearing device also has an auxiliary signal path containing a signal generator for generating a first beat signal. The first beat signal is frequency-shifted relative to a further signal by a beat frequency producing the effect of the monaural or binaural beat for the user during operation. The signal generator is configured such that the first beat signal has, at least in certain operating modes of the hearing device, at least one signal component generated based on the electrical input signal, and hence based on ambient sound, and is denoted as the ambient signal. The beat signal is preferably composed of different signal components, the weighting of which is preferably adjusted depending on a signal level of the input signal.

### CROSS-REFERENCE TO RELATED APPLICATION

This application claims the priority, under 35 U.S.C. § 119, of German Patent Application DE 10 2023 209 094.5, filed Sep. 19, 2023; the prior application is herewith incorporated by reference in its entirety.

### FIELD AND BACKGROUND OF THE INVENTION

The invention relates to an, in particular binaural, hearing system and to a method for generating monaural or binaural beats by using the hearing system.

A hearing system is understood herein to mean a system having at least one hearing device, and in the case of a binaural hearing system to mean a system having two hearing devices. The hearing devices are electronic devices that a user wears on the ear, behind the ear or in the ear. The hearing system is in particular a hearing aid system, which is configured to compensate for a user-specific hearing impairment of a hearing-impaired person.

Such a hearing device usually has an input transducer, which converts an acoustic or even a streamed input signal first into an electrical input signal, which is subsequently conditioned by using an, in particular digital, signal processing unit into an electrical output signal, which is fed to an output transducer, that converts the electrical output signal into an acoustic output signal, which is emitted to the ear of the hearer.

Monaural beats or binaural beats refer to known effects of sensory perception in the human brain, in which the user perceives a pulsating tone (beat) when two identical acoustic signals that differ slightly in frequency from each other are presented to the user. In the case of binaural beats, the user is presented with the two mutually frequency-shifted acoustic signals on two channels, namely at the left ear and at the right ear. In the case of monaural beats, the two frequency-shifted acoustic signals are presented just on one channel.

Certain training effects for the brain can be achieved with such monaural or binaural beats.

### SUMMARY OF THE INVENTION

It is accordingly an object of the invention to provide a hearing system and a method for generating monaural or binaural beats, which overcome the hereinafore-mentioned disadvantages of the heretofore-known systems and methods of this general type and which generate acoustic output signals that are suitable for producing a pleasant perception of monaural or binaural beats for the user.

With the foregoing and other objects in view there is provided, in accordance with the invention, a hearing system which is in particular a hearing aid system that has a user-specific configuration and is configured to compensate for a user-specific hearing impairment.

The hearing system has at least one hearing device and preferably, specifically in the embodiment, two hearing devices as a binaural hearing system, wherein:
the at least one hearing device has a main signal path containing an input transducer for generating an electrical input signal from an, for example, acoustic or streamed input signal, containing a, in particular digital, signal processing unit for processing the electrical input signal and for generating an electrical output signal, and containing an output transducer, which generates an acoustic output signal on the basis of the electrical output signal;
the at least one hearing device also has an auxiliary signal path, which contains in each case a signal generator for generating a first beat signal, wherein the first beat signal is frequency-shifted with respect to a further signal by a beat frequency in such a way that the effect of the monaural or binaural beat is produced for the user during operation;
the at least one hearing device has a summator, which adds at least the first beat signal onto a signal in the main signal path, so that the acoustic output signal is formed by a normal audio signal portion and a beat signal portion; and
the signal generator (28) is connected to the first signal path (8) and is configured in such a way that the first beat signal (B1) has, at least in certain operating modes of the hearing device (4A, 4B), at least one signal component that is generated on the basis of the electrical input signal (E1) and is denoted as the ambient signal.

Advantages and preferred embodiments mentioned with regard to the hearing system can be applied analogously also to the method, and vice versa.

In the case of a binaural hearing system, the two hearing devices each have a communication unit, which forms a communication interface, via which the two hearing devices can mutually communicate and exchange data with each other in a known manner. In particular, a Bluetooth connection is configured for this purpose.

For a hearing system having two hearing devices, in particular for a binaural hearing system, the second hearing device likewise has a main signal path containing the components listed above.

In a preferred embodiment, although not necessarily, such a second hearing device also has an auxiliary signal path, in which is likewise disposed a signal generator for generating a second beat signal. In this embodiment variant, the two beat signals are frequency-shifted with respect to each other by the beat frequency. The second hearing device also has a summator in this case, which adds the second beat signal onto a signal in the first signal path. As a result, in the second hearing device is also generated an acoustic output signal formed by a normal audio signal portion and a beat signal portion based on the second beat signal.

A normal audio signal portion refers to that signal portion in the acoustic output signal that would occur without the addition of the beat signal.

Beat frequency is understood to mean here the frequency by which the two beat signals are frequency-shifted with respect to each other. The beat frequency typically lies in the range of 0.5 Hz to 60 Hz, preferably up to a maximum of 55 Hz and often in the region between 3 Hz and 30 Hz.

For generating the frequency-shifted beat signal, at least the one signal generator has a frequency-shifter, which thus shifts a signal generated or provided by the signal generator (e.g. ambient signal, sinusoidal signal, narrowband noise, signal portion) by the beat frequency, thereby generating the frequency-shifted beat signal.

Using the ambient signal, i.e. the acoustic input signal detected by the input transducer, as the basis for generating the (frequency-shifted) first beat signal achieves the advantage that the ambient sound is used as the basis for generating the beat signals and hence for producing the perception of the beats. The user finds this particularly pleasant because in this case there are no additional, foreign signal components included. Yet at the same time, the desired perception of the monaural or binaural beats is still achieved.

The signal waveform of the ambient signal here corresponds to the signal waveform at least of a selected frequency band of the input signal.

The embodiment in which there is an auxiliary signal path in the second hearing device is not absolutely necessary, even in the case of binaural hearing devices and/or when generating binaural beats.

In the case of generating monaural beats, a normal signal portion based on the input signal and a frequency-shifted signal portion based on the input signal are provided, for example solely within the first hearing device, as the beat signals, which then lead to the perception of the monaural beats. Even when generating binaural beats, in one embodiment variant it is sufficient if, for instance in the signal generator of the first hearing device, the first beat signal is generated on the basis of the input signal merely by frequency-shifting the input signal or signal portions of the input signal and is provided as the ambient signal. The binaural effect is produced, for example, by the audio signal (acoustic output signal) at the second hearing device and by the frequency-shifted beat signal portion (based on the first beat signal) in the acoustic output signal of the first hearing device.

In particular in such variants, an auxiliary signal path is omitted in the second hearing device, and/or a frequency-shifted beat signal is generated solely in the first hearing device.

The preferred variant, however, is that in which both hearing devices have a signal auxiliary path and a signal generator each. In this case, therefore, in both hearing devices the beat signals generated by the respective signal generators have at least the ambient signal as a signal component. In one variant, the beat signals are formed of the ambient signal. The beat signals, and hence also the ambient signals of the two beat signals, are identical in this case except for the frequency shift by the beat frequency. In some variants, the two beat signals/ambient signals can also differ in amplitude.

When the term beat signal is used here, it refers generally to a signal that contributes in the acoustic output signal to a signal portion that contributes to producing the desired effect of the monaural or binaural beat. At least in some specific operating modes of the hearing device, these signal portions (ambient signal) are based on the ambient sound, i.e. on the acoustic input signals.

According to a preferred embodiment, the at least one hearing device has an analysis unit for analyzing the input signal, and the beat signal is composed of one or more signal components. The signal generator is configured in this case to compose the beat signal on the basis of the analysis of the input signal.

The different signal components from which the beat signal is composed depending on the analysis of the input signal and hence on the ambient sound are preferably selected from the ambient signal, a sinusoidal signal and a, in particular narrowband, noise signal. Each of these signals therefore forms a possible signal portion of the particular beat signal.

A narrowband noise signal is understood to mean here noise that extends over a frequency band that preferably has only a bandwidth of 100 Hz to 300 Hz. The noise signal preferably lies between 250 Hz to 5 kHz.

These additional signals besides the ambient signal can also be referred to generally as carrier signals, which are independent of the input signal.

Preferably, the respective signal generators have for each signal component an individual generator part configured for the signal component.

The selection of the signal portions and the composition of the beat signal is made preferably depending on a signal level of the (acoustic) input signal. Each beat signal can be formed of just one signal component, of a plurality of signal components, or even of all the signal components.

This embodiment is based on the finding that for different signal levels of the input signal, different types of beat signals are particularly pleasant for the user.

For instance, it has been found that for low signal levels, a single tone as the beat signal based on a sinusoidal signal is felt to be more annoying or unpleasant than, in particular narrowband, noise. As the signal level increases, this situation is reversed and the sinusoidal signal is felt to be more pleasant. Finally, at higher signal levels, the use of ambient signals is felt to be particularly pleasant because otherwise very loud sinusoidal signals, for example, would have to generated as the additional signals, which would then be felt to be annoying.

The signal level of the input signal is in general a specific value of the signal level, i.e. of the amplitude of the acoustic or else the streamed input signal detected by the input transducer. The (instantaneous) signal level is determined regularly on the basis of an evaluation of the converted electrical input signal. In particular, the determining of the signal level involves an estimate. Thus the determining of the signal level is used to determine or estimate overall a signal level, and hence an amplitude, of the instantaneous ambient sound.

Various methods can be used to determine the signal level. For example, a wideband signal portion of the input signal, for instance the entire bandwidth of the audible frequency spectrum, or just a subband, can be evaluated. In this process, for example, the mean of the signal levels in different frequency bands is found, and the mean signal level is treated as the signal level of the input signal. Alternatively, the frequency band containing the highest signal level is used, and this maximum signal level is treated as the signal level of the input signal.

There is also the option to define a reference frequency, at which the signal level is determined as the reference level and hence as the signal level of the input signal. For example, the reference frequency is a center frequency of a selected frequency band or the frequency having the highest signal level. The frequency band and/or the reference frequency are fixed, for example, or alternatively are each determined dynamically from the instantaneous maximum signal level.

In an expedient development, it is further provided that a different weighting of the signal components is made depending on the ascertained signal level. This means that the weighted composition of the beat signal varies with the signal level.

The weighting is preferably understood to mean the signal level of the respective signal components in the acoustic output signal. If the particular signal component extends over a frequency band, for example in the case of the noise or the ambient signal, then the weighting is determined by the maximum signal level within the frequency band, or alternatively by a mean signal level within the frequency band.

The weighting, and thus the signal levels, of the different signal components is adjusted in particular by using at least one adjustable amplifier element.

The amplifier element preferably has an amplifier sub-component for each of the signal components in order to be able to adjust the individual weighting factors, and thus gains, independently of each other. Expediently, in each individual generator part configured for a particular signal component is assigned an associated amplifier sub-component.

Based on the above-described finding relating to the effect of the different signal components depending specifically on the signal level of the input signal, it is provided in particular that the weighting of the ambient signal increases with increasing signal level.

Alternatively or additionally, at a low signal level, the weighting of the ambient signal is lower than that of the further signal components, and in particular than each of the other signal components. As the signal level increases, the weighting of the ambient signal increases and ultimately exceeds each of the other signal components, for instance in an intermediate level range.

In a preferred embodiment, the noise signal is weighted the most for a low, soft level range of the signal level of the input signal, the sinusoidal signal the most in an intermediate level range, and the ambient signal the most for a high level range. This results overall in a qualitative weighting variation in which the, in particular narrowband, noise dominates for a low signal level, then the sinusoidal signal for an intermediate level range, and finally the ambient signal for a high level range. A low signal level refers in particular to a signal level up to 50 dBSPL or else up to 55 dBSPL (decibel Sound Pressure Level). An intermediate signal level refers to a level range between 50/55 dBSPL-75 dBSPL or even up to 80 dBSPL, and a loud signal level refers in particular to a level above 75 dBSPL or even above 80 dBSPL.

In a preferred embodiment, the analysis is performed repeatedly and in particular continuously and successively. A time interval between two successive analysis steps is preferably less than 300 ms and, for example, equals 100 ms. The time interval in general lies preferably between 100 ms and 300 ms. On the basis of each of these continuously and repeatedly performed analyses, the composition of the beat signal is then adapted depending on the instantaneous analysis, i.e. the composition of the beat signal, and hence the weighting of the different signal components, varies over time. Therefore effectively the composition continuously tracks the variation in level of the input level.

In addition to the analysis and determination of the signal level of the input signal, further parameters are preferably used as the basis for determining the composition of the beat signal. Such parameters are, for example, instantaneous state values of the particular user, for instance heartbeat, movements or accelerations of parts of the body, etc. These parameters are optionally derived by an auxiliary device or else indirectly from the input signals. This embodiment is based on the idea that also in certain state situations of the user, for example when sleeping, exercising, etc., different signal components vary in their suitability for the desired effect.

In an expedient embodiment, the ambient signal is a bandpass-filtered ambient signal. The hearing device accordingly also has a suitable bandpass filter, so that the ambient signal is filtered out of the detected input signal via this filter and has a preset bandwidth. This is based on the idea that only a specific frequency range is particularly suitable for producing the perception of the beats.

The bandpass-filtered ambient signal preferably has a bandwidth in the range of 100 Hz to 400 Hz. In addition, the ambient signal preferably lies in an intermediate band region between 200 Hz and 1000 Hz, and in particular has the aforementioned bandwidth.

According to a preferred development, parameters of the bandpass-filtered ambient signal such as bandwidth, center frequency or lower or upper band frequency, are set depending on a user-specific audiogram, i.e. depending on a user-specific hearing impairment. In particular, the bandwidth and/or the center frequency is selected in such a way that it is outside a frequency band in which the hearing impairment of the user is large compared with other frequency bands and where high amplification is required. The frequency band of the ambient signal is therefore placed in particular in such a frequency range in which relatively low user-specific amplification is required.

In an expedient embodiment, the hearing system is configured in such a way that the values of the parameters of the bandpass-filtered ambient signal such as bandwidth, center frequency or lower or upper band frequency vary over time and in particular depending on the instantaneous input signal. For example, the parameter values are selected depending on the signal level. In particular, for example, a center frequency of the ambient signal is selected in such a way that it lies outside a frequency or a frequency band having the highest signal level of the input signal. This serves to avoid masking effects.

In summary, the bandpass-filtered ambient signal is thus a time-dynamic ambient signal, the parameter values of which vary, and in particular vary depending on the input signal, specifically depending on the signal level of the input signal.

In a preferred development, in each hearing device an adjustment apparatus is preferably assigned to the associated signal generator, which adjustment apparatus includes the adjustable amplifier element described above. The aforementioned analysis unit is also part of this adjustment apparatus. In addition, the adjustment apparatus is configured to adjust a signal level at least of the first beat signal and preferably of both beat signals depending on a current instantaneous signal level of the input signal.

As a result of this measure, the signal level of the beat signal is updated, in particular continuously, to track the instantaneous signal level of the input signal. The analysis and associated adjustment of the signal level of the beat signals is preferably performed at time periods of less than 500 ms and, for example, every 100 ms.

The updating of the signal level of the beat signal results in a particularly pleasant perception of the monaural or binaural beats.

In a preferred embodiment, the hearing system has a user-specific adjustment control, via which it is possible to adjust manually the beat signal, in particular the signal level of the beat signal. For this purpose, the hearing system has, for example, an operating interface, in particular in the form of a portable device such as a smartphone, on which an application is installed that can be used to make adjustments to the hearing device concerned. This portable device is part of the hearing system. Alternatively or additionally, operating controls, which can be used to make the adjustment, can also be disposed directly on at least one hearing device.

Therefore, these manual adjustment facilities provide the user with a device for personally adjusting, at least readjusting, parameters that influence the beats in order to obtain as pleasant a hearer experience as possible. The parameters are in particular the beat frequency and/or the gain for the amplifier element or the signal level of the particular beat signal, in particular the difference in level in comparison with the normal audio signal portion in the acoustic output signal.

In a preferred development, the hearing system has a state identifier, i.e. is configured to identify automatically an instantaneous state of the user. In addition, the hearing system, in particular the adjustment apparatus, is configured to adjust the at least one beat signal depending on the identified instantaneous state. A state of the user refers in particular to an instantaneous bodily state of the user, which is ascertained, for example, by determining the instantaneous pulse of the user and/or by accelerometers. The determining of the instantaneous state can be carried out either using auxiliary devices or directly also by the hearing device. For example, accelerometers can be integrated in the hearing device, or the hearing device is configured to determine from acoustic signals, e.g. structure-borne signals, the instantaneous pulse of the hearing-device wearer.

In particular, a sleep mode is identified, in particular in contrast to an awake mode, and then the parameters are set differently for the different modes. Parameters for the beat signal again are understood to mean in particular the beat frequency (i.e. the magnitude of the frequency shift between the beat signals) and/or the signal level. For instance, when a sleep mode is identified, the signal level is reduced compared with an awake mode. Alternatively or additionally, different beat frequencies are set for the awake mode and the sleep mode. This is because it is known that different beat frequencies have different impacts and training effects on the brain.

In a preferred embodiment, the beat frequency is set, for example in sleep mode, to a range between 35 Hz and 45 Hz and in particular to 40 Hz. Studies have shown that such beat frequencies have particular advantages especially in sleep mode.

Other features which are considered as characteristic for the invention are set forth in the appended claims.

Although the invention is illustrated and described herein as embodied in a hearing system and a method for generating monaural or binaural beats, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a simplified block-diagram representing an embodiment variant of a binaural hearing system for explaining the updating of a signal level of beat signals depending on the signal level of an input signal;

FIG. 2 is an extract of a representation based on the hearing system of FIG. 1 for explaining the composition of the beat signals from different signal components; and

FIG. 3 is a diagram representing a qualitative variation in the weighting of the different signal components as a function of the signal level of the input signal.

### DETAILED DESCRIPTION OF THE INVENTION

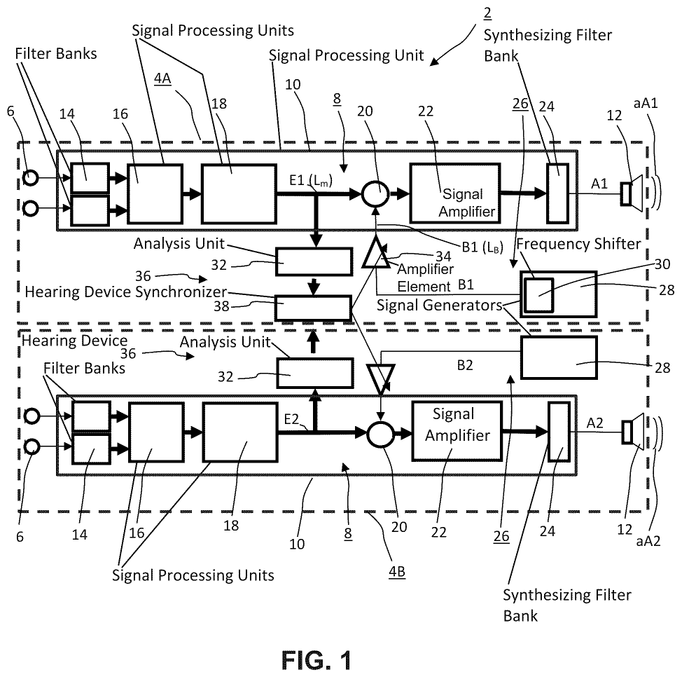

Referring now to the figures of the drawings in detail and first, particularly, to FIG. 1 thereof, there is seen a hearing system 2 which is a hearing aid system configured to compensate for a user-specific hearing impairment. The hearing system 2 is a binaural hearing system having two hearing devices 4A, 4B, which communicate with each other in a manner known per se via a wireless communication unit, in particular a Bluetooth connection unit, not presented in greater detail therein. The two hearing devices 4A, 4B are at least largely identical in construction. Differences will be explicitly addressed.

Each hearing device 4A, 4B has one or more input transducers 6, in particular microphones, through the use of which an input signal, in particular an acoustic ambient signal, is converted into an electrical input signal E1, E2. This electrical input signal is first conditioned in a main signal path 8 and in a signal processing unit 10 in a manner known per se, in particular on the basis of a user-specific audiogram and thus in accordance with a user-specific hearing impairment. The conditioned electrical signal is fed as an electrical output signal A1, A2 to an output transducer 12, which converts the electrical output signal into an acoustic output signal aA1, aA2. The output transducer 12 is in particular a loudspeaker.

The signal processing unit 10 has, for example, a filter bank 14 on its input side, through the use of which the electrical input signal E1, E2 is split into different frequency bands for the subsequent signal processing. Next are usually provided a plurality of signal processing units 16, 18, for instance a beam-former for producing directionality or a noise suppressor for reducing disturbing ambient sound. An electrical input signal E1, E2, which has been partly conditioned by the signal processing units 16, 18, is then fed to a summator 20. Following the summator 20 is a signal amplifier 22, typically containing an integrated compression unit. Finally, after the signal amplifier 22, there is provided, for example, a synthesizing filter bank 24, which re-combines the individual frequency bands and at its end is output the electrical output signal A1, A2.

Each hearing device 4A, 4B has, in addition to the main signal path 8, an auxiliary signal path 26. This auxiliary signal path 26 has a signal generator 28 for generating a beat signal B1, B2. A further signal, in particular in addition to the electrical input signal E1, E2, is provided by the signal generator 28. This further signal is in particular a signal that is independent of the input signal, specifically a sinusoidal signal or an, in particular narrowband, noise signal. There is also the option to use as the basis for the beat signal B1, B2 a signal portion of the electrical input signal E1, E2 acquired by the input transducer 6, i.e. ambient sound. The beat signal B1, B2 is composed, for example, of a plurality of signal portions, which are formed on the basis of the aforementioned signal(s) (sinusoidal signal, noise signal, ambient sound).

A frequency-shifter 30 is integrated in at least one of the two signal generators 28, which is the signal generator in the first hearing device 4A in the exemplary embodiment. The frequency-shifter 30 is used to shift by a beat frequency the previously generated signal (e.g. sinusoidal signal, noise signal). Thus, the two beat signals B1, B2 are two identical signals containing the same signal portions merely shifted with respect to each other by the beat frequency.

In addition, each hearing device 4A, 4B has an analysis unit 32, which is configured to ascertain, in particular estimate, an instantaneous signal level Lₘ of the (acoustic) input signal. For this purpose, the analysis unit 32 evaluates in a suitable manner the electrical input signal E1, E2, which may be partially conditioned and which is present in front of the summator 20.

Depending on the instantaneous, currently present signal level Lₘ ascertained thereby, an instantaneous gain is determined by which the associated beat signal B1, B2 is amplified. An amplifier element 34 is provided for each amplification. The analysis unit 32 and the amplifier element 34 are parts of an adjustment apparatus 36, through the use of which an associated signal level L_{B} for the respective beat signals B1, B2 is adjusted.

According to a preferred embodiment, as is shown in the figure, at least in one of the hearing devices 4A, 4B a synchronizer 38 is provided, which is connected to the two analysis units 32, from which it receives information about the respective signal levels Lₘ, The transfer of this information from the one hearing device 4B to the synchronizer 38 disposed in the other hearing device 4A is performed in particular via the previously described communication interface of the binaural hearing system 2.

The synchronizer 38 evaluates, in particular compares, the two instantaneous signal levels Lₘ, and, on the basis of this evaluation, ascertains suitable synchronized gains for the two beat signals B1, B2. In the case of widely different instantaneous signal levels Lₘ, the synchronizer 38 in particular limits the differences in the signal levels L_{B} of the beat signals B1, B2.

In an alternative, simplified exemplary embodiment, which is not presented therein in greater detail, each adjustment apparatus 36 of the two hearing devices 4A, 4B determines individually an associated instantaneous gain for the respective beat signals B1, B2. This variant therefore dispenses with the synchronizer 38 and synchronization.

The beat signal B1, B2 amplified in each case is then fed to the summator 20. The resultant combined signal, which includes as signal portions the partially conditioned input signal E1, E2 and the beat signal B1, B2, is subsequently fed to the signal amplifier 22, is amplified there, and finally, after the synthesizing filter bank 24, is provided as the outgoing electrical output signal A1, A2, which is then converted into the acoustic output signal aA1, aA2 by the output transducer 12.

The acoustic output signals aA1, aA2 are therefore each composed of a normal audio signal portion and a beat signal portion. The normal audio signal portion is the signal portion that would arise without the associated signal portion of the beat signal. The beat signal portions in the acoustic output signal aA1, aA2 are frequency-shifted with respect to each other by the beat frequency and may have different amplitudes, but are otherwise identical.

The user is thereby provided at both ears with, in addition to the normal audio signal, two (acoustic) beat signals B1, B2, mutually offset by the beat frequency, with the desired result that the user perceives the binaural beats.

The instantaneous signal level Lₘ is detected preferably continuously and preferably at time intervals of less than 300 ms and, for example, every 100 ms, and the signal level L_{B} of the beat signals B1, B2 is continuously updated on the basis of the detected instantaneous signal level Lₘ. This is done preferably in such a way that in the acoustic output signal aA1, aA2, a higher signal level L_{B} of the beat signal portions is established compared with the signal levels Lₘ of the normal audio signal portion. The signal level L_{B} of the beat signal portions is preferably higher by 1 dB to 5 dB and in particular by 3 dB.

The electrical input signals are denoted herein throughout the main signal path 8 uniformly by the reference signs E1, E2, regardless of their processing status.

The further (beat) signals provided by the respective signal generators 28 are denoted herein throughout the signal path, and hence in different processing stages, uniformly by the respective reference signs B1, B2.

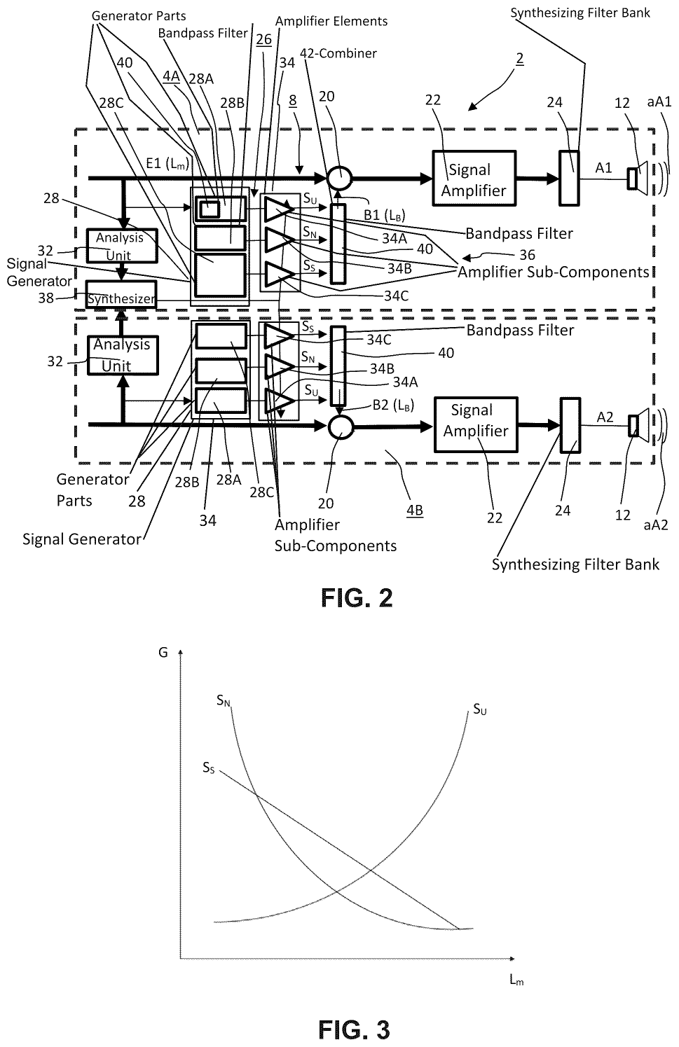

FIG. 2 shows a representation of the hearing system 2 as a magnified extract in the region of the auxiliary signal path 26.

In the embodiment shown in FIG. 2, the beat signals B1, B2 are composed from different signal components. A weighting G of the different signal components is made therein depending on the signal level Lₘ of the acoustic input signal.

The signal generator 28 is first divided into a plurality of generator parts 28A, 28B and 28C for this purpose. In addition, the amplifier element 34 is also divided into a plurality of individually adjustable amplifier sub-components 34A, 34B and 34C. The various generator parts 28A to 28C are configured to generate different signals as the signal components. These are in particular an ambient signal S_{U}, a sinusoidal signal S_{S} and a narrowband noise signal S_{N}.

The generator part 28A serves to generate the ambient signal S_{U}. For this purpose, the electrical input signal E1, E2 is applied on its input side. For example, it has an integrated bandpass filter 40 in order to extract from the input signal E1, E2 a narrowband signal part that then forms the ambient signal S_{U}.

The generator parts 28A-28C of the two hearing devices 4A, 4B are identical in configuration with the difference that in the generator parts 28A to 28C of the first hearing device 4A a frequency-shifter 30 is also additionally integrated, but is not shown explicitly therein, in order to generate the desired frequency shift by the beat frequency.

The generator parts 28B, 28C each generate autonomous signals independently of the electrical input signal E1, E2.

The different signal components S_{U}, S_{N}, S_{S} are amplified individually by using the respective amplifier sub-components 34A to 34C, which are each assigned to an associated generator part 28A to 28C. They are subsequently combined in a combiner 42, which in particular is in the form of a summator, and then form the corresponding beat signal B1, B2, which is fed to the summator 20 in the main signal path 8.

The individual gains for the different signal components S_{U}, S_{N}, S_{S} are adjusted depending on the signal level L_{M} of the input signal. For this purpose, as already described in relation to FIG. 1 an analysis unit 32, which is configured to determine and in particular estimate the incoming signal level L_{M}, is integrated in each hearing device 4A, 4B.

FIG. 2 shows, like FIG. 1, an embodiment variant containing the synchronizer 38. This is again used for synchronized control of the amplifier element 34. The basic principles are the same as described with regard to FIG. 1. Unlike FIG. 1, however, the amplifier sub-components 34A to 34C are now each controlled individually, so that different gains and hence weightings G are set for the different signal components S_{U}, S_{N}, S_{S}. This means that the different signal components S_{U}, S_{N}, S_{S} are weighted differently in the subsequently combined beat signal B1, B2. The signals B1, B2 then each have a signal level L_{B}. The gains for the signal components S_{U}, S_{N}, S_{S} are suitably selected to achieve the desired value for the signal level L_{B}, as was described in particular in connection with FIG. 1.

In an alternative embodiment, the synchronizer 38 is omitted and the adjustment of the weightings G is performed in the two hearing devices, for instance independently of each other and each on the basis of the signal level Lₘ ascertained by the analysis unit 32 of the associated hearing device 4A, 4B.

FIG. 3 shows the qualitative variation in the weighting of the different signal components S_{U}, S_{N}, S_{S} as a function of the signal level of the input signal Lₘ. The signal level L_{M} is given on the x-axis (increasing from left to right), and weighting G and hence the amplification of the different signal components S_{U}, S_{N}, S_{S} is shown on the y-axis.

As is clearly evident therein, at a low, soft signal level Lₘ, the noise signal S_{N} dominates, the weighting G of which decreases with increasing signal level Lₘ, preferably in the manner of a hyperbola, which approaches asymptotically a minimum value, which is preferably 0 or alternatively is not equal to 0.

At the low, soft signal level Lₘ, the sinusoidal signal S_{S} also has, in comparison with the ambient signal S_{U}, a high weighting factor, but which is less than that of the noise signal S_{N}. The weighting factor of the sinusoidal signal S_{S} decreases, preferably linearly, with increasing signal level Lₘ.

In general, the noise signal S_{N} decreases more sharply with increasing signal level Lₘ than the sinusoidal signal S_{S}, with the result that their two curves intersect and therefore the sinusoidal signal S_{S} dominates in an intermediate level range.

In contrast with these two signal components S_{N}, S_{S}, the ambient signal S_{U} increases with increasing signal level Lₘ. For example, the rise follows an exponential curve.

In the low, soft level range, the ambient signal S_{U} starts at a constant value, in particular at 0, and therefore, in the soft level range, the beat signal B1, B2 is composed solely of the two other signal components S_{S}, S_{N}.

In the intermediate level range, in contrast, each beat signal B1, B2 is composed of all three signal components S_{U}, S_{N} S_{S}, with the weighting varying within the intermediate level range. The point of intersection of the weighting curves of the signal components S_{S}, S_{N} is regarded herein as the start of the intermediate level range.

In the intermediate level range, the weighting of the ambient signal S_{U} increases progressively, with it first exceeding, for example, the weighting of the noise signal S_{N} and later also the weighting of the sinusoidal signal S_{S}. The signal level Lₘ at, and above, which the largest weighting portion is formed by the ambient signal S_{U} is herein defined as the end of the intermediate level range. In the present case it is the point of intersection between the weighting curve of the sinusoidal signal S_{S} and of the ambient signal S_{U}.

In the high level range, which is adjacent to the intermediate level range, the weighting G of the ambient signal S_{U} continues to increase, while the weighting of the two other signals S_{N}, S_{S} decreases further and in particular even returns to 0. Thus, in high level ranges, the ambient signal S_{U} predominates (greater than 75% of the total level) or even is solely present, and therefore each beat signal B1, B2 is formed predominantly or entirely by the ambient signal S_{U}.

The different weightings of the different signal components S_{U}, S_{N}, S_{S} depending on the signal level Lₘ is based on the finding that the different signal components S_{U}, S_{N}, S_{S} are perceived differently depending on the signal level Lₘ. For instance, studies have found that at low signal levels, a pure sinusoidal tone is felt to be unpleasant, whereas in an intermediate level range, this is felt to be more pleasant than a noise signal. At high level ranges, using the ambient signal S_{U} as a signal component has proved particularly advantageous.

The units mentioned above such as filter bank 14, 24, signal processing unit 16, 18, summator 20, signal amplifier 22, signal generator 28 having generator parts 28A-28C, frequency-shifter 30, analysis unit 32, amplifier element 34 having amplifier sub-components 34A-34C, synchronizer 38 and bandpass filters 40 are each electronic components or groups of electronic circuit components, which during operation implement the desired functions described. For example, these units are integrated in one or more integrated circuits, microchips and/or ASICs.

The following is a summary list of reference numerals and the corresponding structure used in the above description of the invention:
2 hearing system
4A, B hearing device
6 input transducer
8 main signal path
10 signal processing unit
12 output transducer
14 filter bank
16 signal processing unit
18 signal processing unit
20 summator
22 signal amplifier
24 synthesizing filter bank
26 auxiliary signal path
28 signal generator
28A-C generator parts
30 frequency-shifter
32 analysis unit
34 amplifier element
34A-C amplifier sub-component
36 adjustment apparatus
38 synchronizer
40 bandpass filter
E1, E2 electrical input signal
A1, A2 electrical output signal
aA1, aA2 acoustic output signal
B1, B2 beat signal
Lₘ signal level of the input signal
L_{B} signal level of the beat signal
S_{U} ambient signals
S_{S} sinusoidal signal
S_{N} noise signal
G weighting

1. A monaural or binaural hearing system for generating monaural or binaural beats, the hearing system comprising:
at least one hearing device having a main signal path, said main signal path containing an input transducer for generating an electrical input signal from an input signal or from an acoustic input signal, said main signal path containing a signal processing unit for processing the electrical input signal and for generating an electrical output signal, and said main signal path containing an output transducer generating an acoustic output signal based on the electrical output signal;
said at least one hearing device having an auxiliary signal path, said auxiliary signal path containing a signal generator for generating a first beat signal, the first beat signal being frequency-shifted relative to a further signal by a beat frequency, producing an effect of a monaural or binaural beat for a user during operation;
said at least one hearing device having a summator adding at least the first beat signal onto a signal in said main signal path; and
said signal generator being connected to said main signal path and configured to cause the first beat signal to have, at least in certain operating modes of the hearing device, at least one signal component being generated based on the electrical input signal and denoted as an ambient signal.

2. The hearing system according to claim 1, which further comprises:
a second hearing device forming a binaural hearing system with said at least one hearing device for generating binaural beats, said at least one hearing device being a first hearing device, and said first hearing device and said second hearing device being configured for wireless binaural communication with each other;
said second hearing device having a main signal path, said main signal path containing an input transducer for generating an electrical input signal, said main signal path containing a signal processing unit for processing the input signal and for generating an electrical output signal, and said main signal path containing an output transducer generating an acoustic output signal based on the electrical output signal;
said second hearing device having an auxiliary signal path, said auxiliary signal path containing a signal generator for generating the further beat signal;
said second hearing device having a summator adding the beat signals onto a signal in said main signal path; and
said signal generator being connected to said main signal path and configured to cause the beat signal to have, at least in certain operating modes of the hearing device, at least one signal component being generated based on the electrical input signal and denoted as an ambient signal.

3. The hearing system according to claim 2, wherein at least one of said hearing devices has an analysis unit for analyzing the input signal, the beat signal is composed of one or more signal components, and said signal generator is configured to compose the beat signal based on the analysis of the input signal.

4. The hearing system according to claim 3, wherein the signal components are selected from a sinusoidal signal, a noise signal, a narrowband noise signal and the ambient signal derived from the electrical input signal.

5. The hearing system according to claim 4, wherein said analysis unit is configured to analyze the signal level of the input signal, and the composition of the beat signal is made based on the analysis of the input signal.

6. The hearing system according to claim 5, wherein a different weighting of the signal components is made depending on the signal level.

7. The hearing system according to claim 6, wherein the weighting of the ambient signal increases with increasing signal level.

8. The hearing system according to claim 7, wherein at low signal levels, the weighting of the ambient signal is lower than the weighting of each of the other signal components.

9. The hearing system according to claim 7, wherein the noise signal is weighted the most in a low level range of the signal level, the sinusoidal signal is weighted the most in an intermediate level range, and the ambient signal is weighted the most in a high level range.

10. The hearing system according to claim 5, wherein the analysis is performed repeatedly, and said signal generator is configured to adapt the composition of the beat signal depending on an instantaneous analysis.

11. The hearing system according to claim 6, wherein, in addition to the signal level, further parameters are used as assessment criteria for the composition of the beat signal and the weighting of the signal components.

12. The hearing system according to claim 2, wherein at least one of said hearing devices has a bandpass filter for generating a bandpass-filtered ambient signal.

13. The hearing system according to claim 1, wherein the ambient signal has a bandwidth in a range of 100 Hz to 400 Hz.

14. The hearing system according to claim 12, wherein the ambient signal lies in an intermediate band region between 200 Hz and 1000 Hz.

15. The hearing system according to claim 12, wherein parameters of the bandpass-filtered ambient signal, including bandwidth, center frequency or lower or upper band frequency, are set depending on a user-specific audiogram.

16. The hearing system according to claim 12, wherein parameters of the bandpass-filtered ambient signal, including bandwidth, center frequency or lower or upper band frequency, vary over time and are dependent on an instantaneous input signal.

17. The hearing system according to claim 15, wherein said signal generator has an associated adjustment apparatus with an adjustable amplifier element, said adjustment apparatus has an analysis unit for determining an instantaneous signal level of the input signal, and said associated adjustment apparatus is configured to adjust a signal level of the first beat signal depending on an instantaneous signal level of the input signal.

18. The hearing system according to claim 16, wherein said signal generator has an associated adjustment apparatus with an adjustable amplifier element, said adjustment apparatus has an analysis unit for determining an instantaneous signal level of the input signal, and said associated adjustment apparatus is configured to adjust a signal level of the first beat signal depending on an instantaneous signal level of the input signal.

19. The hearing system according to claim 1, wherein the hearing system is configured for manual adjustment of the at least one beat signal by the user.

20. The hearing system according to claim 1, wherein the hearing system is configured to automatically identify an instantaneous state of the user, or a sleep state of the user, and the hearing system is configured to adjust the at least one beat signal depending on the identified instantaneous state.

21. A method for generating monaural or binaural beats in a hearing system, the method comprising:
providing the hearing system with at least one hearing device having a main signal path containing an input transducer generating an electrical input signal from an input signal or from an acoustic input signal, the main signal path containing a signal processing unit processing the electrical input signal and generating an electrical output signal, and the main signal path containing an output transducer generating an acoustic output signal based on the electrical output signal;
providing the at least one hearing device with an auxiliary signal path containing a signal generator generating a first beat signal, the first beat signal being frequency-shifted relative to a further signal by a beat frequency causing an effect of the monaural or binaural beat to be produced for a user during operation;
providing the at least one hearing device with a summator adding at least the first beat signal onto a signal in the first signal path; and
providing the beat signal, at least in certain operating modes of the hearing device, with at least one signal component being generated based on the electrical input signal and being denoted as an ambient signal.
